(19) United States
(12) Patent Application Publication
(10) Pub. No.: US 2006/0269605 A1
(43) Pub. Date: Nov. 30, 2006

(54) Multilayer pharmaceutical dosage form containing a substance that acts in a modulatory manner with regard to the release of active substances

(75) Inventors: Rosario Lizio, Rossdorf (DE); Hans-Ulrich Petereit, Darmstadt (DE); Manfred Assmus, Bickenbach (DE); Hema Ravishankar, Mumbai (IN)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: Roehm Gmbh & Co. KG, Darmstadt (DE)

(21) Appl. No.: 10/572,963

(22) PCT Filed: Sep. 15, 2004
(86) PCT No.: PCT/EP04/10297

§ 371 Date: Mar. 21, 2006

(30) Foreign Application Priority Data
Nov. 13, 2003 (DE) 10353186.6

(51) Int. Cl. A61K 9/24

(52) U.S. Cl. 424/472

(57) ABSTRACT

The invention relates to a multilayer pharmaceutical dosage form for the controlled release of active substances, containing: a) a core layer containing a substance that acts in a modulatory manner with regard to the release of active substances, optionally a neutral core and/or an active substance; b) an inner control layer that influences the release of the substance that acts in a modulatory manner and of the optionally contained active substance from the core layer, containing pharmaceutically useable polymers, waxes, resins and/or proteins; c) an active substance layer containing a pharmaceutical active substance and, optionally, a substance that acts in a modulatory manner; d) an outer control layer containing a (meth)acrylate copolymer or a mixture consisting of a number of (meth)acrylate copolymers comprised of 98 to 85 C₁-C₄ alkyl esters of (meth)acrylic acid and 2 to 15% by weight of methacrylate monomers with a quaternary ammonium group in the alkyl radical and optionally containing pharmaceutically useable polymers that are insoluble in water, whereby the layers can contain, in addition and in a known manner, pharmaceutically conventional adjuvants.

The invention relates to a multilayer pharmaceutical form for controlled active ingredient release.

### PRIOR ART

EP-A 0 463 877 describes pharmaceutical compositions with delayed active ingredient release consisting of a core with an active pharmaceutical ingredient as a monolayer coating film which comprises a water-repellent salt and a water-insoluble copolymer of ethyl acrylate, methyl methacrylate and trimethylammoniumethyl methacrylate chloride. The water-repellent salt may be for example Ca stearate or Mg stearate. Sigmoidal release plots are obtained.

EP-A 0 225 085, EP-A 0 122 077 and EP-A 0 123 470 describe the use of organic acid in medicament cores which are provided with various coatings from organic solutions. Essentially sigmoidal release characteristics result.

EP-A 0 436 370 describes pharmaceutical compositions with delayed active ingredient release consisting of a core with an active pharmaceutical ingredient and an organic acid and an outer coating film which has been applied by aqueous spraying and is a copolymer of ethyl acrylate, methyl methacrylate and trimethylammoniumethyl methacrylate chloride. In this case, sigmoidal release plots are likewise obtained.

WO 00/19984 describes a pharmaceutical preparation consisting of (a) a core comprising an active ingredient, where appropriate a carrier and conventional pharmaceutical additives, and the salt of an organic acid whose proportion in the weight of the core amounts to 2.5 to 97.5% by weight, and (b) an outer coating film which consists of one or more (meth)acrylate copolymers and, where appropriate, of conventional pharmaceutical excipients, where 40 to 100% by weight of the (meth)acrylate copolymers consist of 93 to 98% by weight of free-radical polymerized C₁ to C₄ alkyl esters of acrylic or methacrylic acid and 7 to 2% by weight of (meth)acrylate monomers with a quaternary ammonium group in the alkyl radical and may where appropriate be present in a mixture, with 1 to 60% by weight of one or more further (meth)acrylate copolymers which are different from the first-mentioned (meth)acrylate copolymers and are composed of 85 to 100% by weight of free-radical polymerized C₁ to C₄ alkyl esters of acrylic or methacrylic acid and, where appropriate, up to 15% by weight of further (meth)acrylate monomers with basic groups or acidic group in the alkyl radical.

WO 00/74655 describes an active ingredient release system with a double release pulse which is brought about by a three-layer structure. The core comprises an active ingredient and a substance which swells in the presence of water, e.g. a crosslinked polyacrylic acid. An inner coating consists of a water-insoluble carrier material, e.g. a cationic (meth)acrylate copolymer, and comprises a water-soluble particulate material, e.g. a pectin, whereby pore formation can be achieved. An outer coating comprises the same or a different active ingredient. In the gastrointestinal tract there is initial release of the active ingredient located on the outside, while the active ingredient present in the core is released after a time lag through the pores in the middle layer. The three-layer pharmaceutical form may optionally also have a further coating, e.g. composed of a carboxyl group-containing (meth)acrylate copolymer.

U.S. Pat. No. 5,508,040 describes a multiparticulate pharmaceutical form consisting of large number of pellets which are held together in a binder. The pellets have an active ingredient and an osmotically active modulator, e.g. NaCl or an organic acid, in the core. The pellet cores are provided with coatings of different thicknesses, e.g. composed of (meth)acrylate copolymers with quaternary ammonium groups. To reduce the permeability, the coatings also comprise hydrophobic substances, e.g. fatty acids, in amounts of 25% by weight or above. The multiparticulate pharmaceutical form is released through a the contained active ingredient in a large number of pulses which corresponds to the number of pellet populations with coatings of different thicknesses.

EP 1 064 938 A1 describes a pharmaceutical form which has an active ingredient and a surface-active substance (surfactant) in the core. The core may additionally comprise an organic acid and is coated with (meth)acrylate copolymers with quaternary ammonium groups. “Pulsatile” release plots are obtained. Stepped release plots can be obtained by combining pellets with different coatings in one pharmaceutical form.

WO 01/13895 describes bimodal release systems for active ingredients having a sedative hypnotic effect. The release profiles are achieved by mixtures of different pellet populations.

WO 01/37815 describes multilayer release systems for controlled, pulsatile delivery of active ingredients. In this case, an inner membrane which can be dissolved by the active ingredient formulation present in the cores is present. Also present is an outer membrane which additionally has a pore-forming substance.

WO 01/58433 describes multilayer release systems for controlled, pulsatile delivery of active ingredients. In this case, the active ingredient is present in the core and is surrounded by a polymer membrane which is soluble in intestinal juice. An outer membrane consists of a mixture of a polymer which is soluble in intestinal juice with a water-insoluble polymer in defined ranges of amounts. An intermediate layer comprising an organic acid may be present between the inner and outer membrane.

Problem and Solution

Starting from EP-A 0 436 370 and WO 00/19984, it was intended to develop a pharmaceutical form which permits the permeability of film coatings to be influenced by intrinsic modulation so that release profiles with zero order, first order, first order with initial accelerated phase, slow-fast, fast-slow profiles can be adjusted individually depending on the active ingredient and therapeutic requirements.

The problem is solved by a

multilayer pharmaceutical form for controlled active ingredient release, comprising
a) a core layer comprising a substance having a modulating effect in relation to active ingredient delivery, where appropriate a neutral core and/or an active ingredient,
b) an inner controlling layer which influences the delivery of the substance having a modulating effect and of the active ingredient which is present where appropriate from the core layer, consisting of pharmaceutically usable polymers, waxes, resins and/or proteins,
c) an active ingredient layer comprising an active pharmaceutical ingredient and, where appropriate, a substance having a modulating effect,
d) an outer controlling layer comprising at least 60% by weight of one or a mixture of a plurality of (meth)acrylate copolymers composed of 98 to 85 C₁ to C₄ alkyl esters of (meth)acrylic acid and 2 to 15% by weight of methacrylate monomers with a quaternary ammonium group in the alkyl radical, and, where appropriate, up to 40% by weight of further pharmaceutically usable polymers,
   where the layers may additionally and in a manner known per se comprise pharmaceutically usual excipients.
   Implementation of the Invention

The invention relates to a multilayer pharmaceutical form for controlled active ingredient release comprising essentially a core layer a) and layers b), c) and d). It is also possible in addition for usual topcoat layers, which may for example be pigmented, to be present.

The Core Layer a)

The multilayer pharmaceutical form has a core layer a) comprising a substance having a modulating effect in relation to active ingredient delivery, where appropriate a neutral core (nonpareilles) and/or an active ingredient.

Suitable processes for producing the core layer a) are direct compression, compression of dry, wet or sintered granules, extrusion and subsequent rounding off, wet or dry granulation or direct pelleting (e.g. on plates) or by binding powders (powder layering) onto active ingredient-free beads or cores (nonpareilles) or active ingredient-containing particles.

Besides the active ingredient, the substance having a modulating effect in relation to active ingredient delivery, and the neutral core (nonpareilles) which is present where appropriate, the core layer a) may comprise further pharmaceutical excipients: binders such as cellulose and derivatives thereof, polyvinyl-pyrrolidone (PVP), humectants, disintegration promoters, lubricants, disintegrants, starch and derivatives thereof, sugar solubilizers or others.

Alternatives for the Structure of the Core Layer a)

The core layer may alternatively essentially comprise the following ingredients
I. a substance having a modulating effect, e.g. in crystalline, granular or coprecipitate form. The size of granules or crystals may be for example between 0.01 and 2.5 mm,
II. a substance having a modulating effect and an active ingredient, which may be present in successive layers in any sequence or in a mixture,
III. a neutral core (nonpareilles) coated with a substance having a modulating effect,
IV. a neutral core (nonpareilles) coated with a substance having a modulating effect and with an active ingredient, which may be present in successive layers in any sequence or in a mixture.
   Substances having a Modulating Effect

Substances having a modulating effect which are to be used according to the invention may have a molecular weight of below 500, be in solid form and be ionic.

The substance having a modulating effect is preferably water-soluble.

The substance having a modulating effect may be for example an organic acid or the salt of an organic or inorganic acid.

The substance having a modulating effect may be for example succinic acid, citric acid, tartaric acid, laurylsulphuric acid, a salt of these acids or a salt of the following anions: taurochlolate and other cholates, chlorides, acetates, lactates, phosphates and/or sulphates.

Mode of Functioning of the Components with One Another

The mode of functioning of the substance having a modulating effect in the multilayer pharmaceutical form can be described approximately as follows: Na succinate (succinic acid), Na acetate and citric acid increase the rate of active ingredient delivery. NaCl and Na citrate decrease the rate of active ingredient delivery.

If the active ingredient layer c) comprises in addition to the inner core layer a) a substance having a modulating effect, the active ingredient delivery is determined firstly by the substance having a modulating effect which is present in the outer layer, the active ingredient layer c). If this substance is substantially consumed, the effect of the substance having a modulating effect in the inner layer, the inner core layer a), starts and determines further active ingredient release.

The various active ingredient delivery profiles can be adapted to the active ingredient and the therapeutic aim by combining different amounts of one and/or different substances having a modulating effect in the two layers. There is in addition the effect of the inner controlling layer b) which in turn itself controls delivery of the substance having a modulating effect from the core layer a).

The amount of active ingredient delivered is essentially controlled by the outer controlling layer d). If the inner controlling layer additionally comprises an active ingredient, this layer can be used to adjust the active ingredient delivery profile towards the end of active ingredient delivery.

If the active ingredients themselves comprise ionic groups or are present in the salt form, the active ingredient itself can influence the effect of the substance or substances having a modulating effect so that the latter is diminished or enhanced. This interaction can be utilized as further control element. The is the case for example with the active ingredients metoprolol succinate and terbutaline sulphate.

The Inner Controlling Layer b)

The inner controlling layer influences the delivery of the substance having a modulating effect and of the active ingredient which is present where appropriate from the core layer. The inner controlling layer comprises essentially pharmaceutically usable polymers, waxes and/or proteins. To assist the formulation it is possible to admix further pharmaceutically customary excipients such as, for example, binders such as cellulose and derivatives thereof, plasticizers, polyvinylpyrrolidone (PVP), humectants, disintegration promoters, lubricants, disintegrants, starch and derivatives thereof, sugars and/or solubilizers.

The inner controlling layer b) may consist for example of a polymer which is insoluble in water or only swellable in water.

Examples of suitable polymers are the following:

copolymers of methyl methacrylate and/or ethyl acrylate and methacrylic acid, copolymers of methyl methacrylate, methyl acrylate and methacrylic acid, copolymers of methyl methacrylate, butyl methacrylate and dimethylethyl methacrylate, copolymers of methyl methacrylate, ethyl acrylate and trimethylammoniumethyl methacrylate, copolymers of methyl methacrylate and ethyl acrylate, copolymers of ethyl acrylate, methyl acrylate, butyl methacrylate and methacrylic acid,

polyvinylpyrolidones (PVPs), polyvinyl alcohols, polyvinyl alcohol-polyethylene glycol graft copolymer (Kollicoat®), starch and derivatives thereof, polyvinyl acetate phthalate (PVAP, Coateric®), polyvinyl acetate (PVAc, Kollicoat), vinyl acetate/vinylpyrolidone copolymer (Kollidon® VA64), vinyl acetate: crotonic acid 9:1 copolymer (VAC: CRA, Kollicoat® VAC), polyethylene glycols with a molecular weight above 1000 (g/mol), chitosan, a (meth)acrylate copolymer consisting of 20-40% by weight of methyl methacrylate and 60 to 80% by weight of methacrylic acid, a crosslinked and/or uncrosslinked polyacrylic acid, an Na alginate, and/or a pectin,

celluloses such as, for example, anionic carboxymethyl-cellulose and salts thereof (CMC, Na—CMC, Ca—CMC, Blanose, Tylopur), carboxymethylethylcellulose (CMEC, Duodcell®), hydroxyethylcellulose (HEC, Klucel), hydroxypropylcellulose (HPC), hydroxypropylmethyl-cellulose (HPMC, Pharmacoat, Methocel, Sepifilm, Viscontran, Opadry), hydroxymethylethylcellulose (HEMC), ethylcellulose (EC, Ethocel®, Aquacoat®, Surelease®), methylcellulose (MC, Viscontran, Tylopur, Methocel), cellulose esters, cellulose glycolate, cellulose acetate phthalate (CAP, Cellulosi acetas, PhEur, cellulose acetate phthalate, NF, Aquateric®), cellulose acetate succinate (CAS), cellulose acetate trimeliate (CAT), hydroxypropylmethylcellulose phthalate (HPMCP, HP50, HP55), hydroxypropylmethylcellulose acetate succinate (HPMCAS-LF, -MF, -HF).

The inner controlling layer may consist of a wax such as, for example, carnauba wax and/or beeswax, or comprise the latter.

The inner controlling layer may comprise the resin shellac or consist thereof.

The inner controlling layer may comprise a protein such as, for example, albumin, gelatin, zein, gluten, collagen and/or lectins, or consist thereof. The protein of the inner controlling layer should preferably have no therapeutic function, as is the case with protein or peptide active ingredients, so that the technical effects of the inner controlling layer b) on the one hand and of the active ingredient layer c) or of the core layer layer a), if the latter comprises an active ingredient, on the other hand do not overlap where possible.

The Active Ingredient Layer c)

The active ingredient layer c) comprises an active pharmaceutical ingredient which may be identical to or different from the active ingredient of the core layer, and where appropriate a substance having a modulating effect, which may be identical to or different from the substance having a modulating effect of the core layer.

Active Ingredients

The multilayer pharmaceutical form of the invention is suitable in principle for any active ingredients. Medicinal substances in use can be found in reference works such as, for example, the Rote Liste or the Merck Index.

The medicinal substances employed for the purposes of the invention are intended to be used on or in the human or animal body in order
1. to cure, to alleviate, to prevent or to diagnose disorders, conditions, physical damage or pathological symptoms.
2. to reveal the condition, the status or the functions of the body or mental states.
3. to replace active substances or body fluids produced by the human or animal body.
4. to ward off, to eliminate or to render harmless pathogens, parasites or exogenous substances, or
5. to influence the condition, the status or the functions of the body or mental states.

The formulation of the invention is suitable for administration of in principle any active pharmaceutical ingredients or biologically active substances which can preferably be administered as ingredient of a multiparticulate pharmaceutical form, of pellet-containing tablets, minitablets, capsules, sachets, effervescent tablets or powders for reconstitution.

Therapeutic Classes

These pharmaceutically active substances may belong to one or more active ingredient classes such as ACE inhibitors, adrenergics, adrenocorticosteroids, acne therapeutic agents, aldose reductase inhibitors, aldosterone antagonists, alpha-glucosidase inhibitors, alpha 1 antagonists, remedies for alcohol abuse, amino acids, amoebicides, anabolics, analeptics, anaesthetic additions, anaesthetics (non-inhalational), anaesthetics (local), analgesics, androgens, angina therapeutic agents, antagonists, antiallergics, antiallergics such as PDE inhibitors, antiallergics for asthma treatment, further antiallergics (e.g. leukotriene antagonists, antianaemics, antiandrogens, antianxiolytics, antiarthritics, antiarrhythmics, antiatheriosclerotics, antibiotics, anticholinergics, anticonvulsants, antidepressants, antidiabetics, antidiarrhoeals, antidiuretics, antidotes, antiemetics, antiepileptics, antifibrinolytics, antiepileptics, antihelmintics, antihistamines, antihypotensives, antihypertensives, antihypertensives, antihypotensives, anticoagulants, antimycotics, antiestrogens, antiestrogens (non-steroidal), antiparkinson agents, antiinflammatory agents, antiproliferative active ingredients, antiprotozoal active ingredients, antirheumatics, antischistosomicides, antispasmolytics, antithrombotics, antitussives, appetite suppressants, arteriosclerosis remedies, bacteriostatics, beta-blockers, beta-receptor blockers, bronchodilators, carbonic anhydrase inhibitors, chemotherapeutic agents, choleretics, cholinergics, cholinergic agonists, cholinesterase inhibitors, agents for the treatment of ulcerative colitis, cyclooxygenaze inhibitors diuretics, ectoparasiticides, emetics, enzymes, enzyme inhibitors, enzyme inhibitors, active ingredients to counter vomiting, fibrinolytics, fungistatics, gout remedies, glaucoma therapeutic agents, glucocorticoids, glucocorticosteroids, haemostatics, cardiac glycosides, histamine H2 antagonists, hormones and their inhibitors, immunotherapeutic agents, cardiotonics, coccidiostats, laxatives, lipid-lowering agents, gastrointestinal therapeutic agents, malaria therapeutic agents, migraine remedies, microbiocides, Crohn's disease, metastasis inhibitors, migraine remedies, mineral preparations, motility-increasing active ingredients, muscle relaxants, neuroleptics, active ingredients for treatment of estrogens, osteoporosis, otologicals, antiparkinson agents, phytopharmaceuticals, proton pump inhibitors, prostaglandins, active ingredients for treating benign prostate hyperblasia, active ingredients for treating pruritus, psoriasis active ingredients, psychoactive drugs, free-radical scavengers, renin antagonists, thyroid therapeutic agents, active ingredients for treating seborrhoea, active ingredients to counter seasickness, spasmolytics, alpha- and beta-sympathomimetics, tenatoprazole, platelet aggregation inhibitors, tranquilizers, ulcer therapeutic agents, further ulcer therapeutic agents, agents for the treatment of urolithiasis, virustatics, vitamins, cytokines, active ingredients for combination therapy with cytostatics, cytostatics.

Active Ingredients

Examples of suitable active ingredients are acarbose, acetylsalicylic acid, abacavir, aceclofenac, aclarubicin, acyclovir, actinomycin, adalimumab, adefovir, adefovirdipivoxil, adenosylmethionine, adrenaline and adrenaline derivatives, agalsidase alpha, agalsidase beta, alemtuzumab, almotriptan, alphacept, allopurinol, almotriptan, alosetron, alprostadil, amantadine, ambroxol, amisulpride, amlodipine, amoxicillin, 5-aminosalicylic acid, amitriptyline, amlodipine, amoxicillin, amprenavir, anakinra, anastrozole, androgen and androgen derivatives, apomorphine, aripiprazole, arsenic trioxide, artemether, atenolol, atorvastatin, atosiban, azathioprine, azelaic acid, barbituric acid derivatives, balsalazide, basiliximab, beclapermin, beclomethasone, bemiparin, benzodiazepines, betahistine, bexaroten, bezafibrate, bicalutamide, bimatoprost, bosentan, botulinus toxim, brimonidine, brinzolamide, budesonide, budipine, bufexamac, bumetanide, buprenorphine, bupropion, butizine, calcitonin, calcium antagonists, calcium salts, candesartan, capecitabine, captopril, carbamazepine, carifenacin, carvedilol, caspofungin, cefaclor, cefadroxil, cefalexin cefalosporins, cefditoren, cefprozil, celecoxib, cepecitabine, cerivastatim, cetirizine, cetrorelix, cetuximab, chenodeoxycholic acid, chorionic gonadotropin, ciclosporin, cidofovir, cimetidine, ciprofloxacin, cisplatin, cladribine, clarithromycin, clavulanic acid, clindamycin, clobutinol, clonidine, clopidogrel, codeine, caffeine, colestyramine, cromoglicic acid, cotrimoxazole, coumarin and coumarin derivatives, darbepoetin, cysteamine, cysteine, cytarabine, cyclophosphamide, cyproterone, cytarabine, daclizumab, dalfopristin, danaparoid, dapiprazole, darbepoetin, defepripone, desipramine, desirudin, desloaratadine, desmopressin, desogestrel, desonide, dexibuprofen, dexketoprofen, disoproxil, diazepam and diazepam derivatives, dihydralazine, diltiazem, dimenhydrinate, dimethyl sulphoxide, dimeticon, dipivoxil, dipyridarnoi, dolasetron, domperidone, and domperidane derivatives, donepzil, dopamine, doxazosin, doxorubizin, doxylamine, diclofenac, divalproex, dronabinol, drospirenone, drotrecogin alpha, dutasteride, ebastine, econazole, efavirenz, eletripan, emidastine, emtricitabine, enalapril, encepur, entacapone, enfurvirtide, ephedrine, epinephrine, eplerenone, epoetin and epoetin derivatives, eprosartan, eptifibatide, ertapenem, esomeprazole, estrogen and estrogen derivatives, etanercept, ethenzamide, ethinestradiol, etofenamate, etofibrate, etofylline, etonogestrel, etoposide, exemestan, exetimib, famciclovir, famotidine, faropenan daloxate, felodipine, fenofibrate, fentanyl, fenticonazole, fexofenadine, finasteride, fluconazole, fludarabine, flunarizine, fluorouracil, fluoxetine, flurbiprofen, flupirtine, flutamide, fluvastatin, follitropin, fomivirsen, fondaparinux, formoterol, fosfomicin, frovatriptan, furosemide, fusidic acid, gadobenate, galantamine, gallopamil, ganciclovir, ganirelix, gatifloxacin, gefitinib, gemfibrozil, gentamicin, gepirone, progestogen and progestogen derivatives, ginkgo, glatiramer, glibenclamide, glipizide, glucagon, glucitol and glucitol derivatives, glucosamine and glucosamine derivatives, glycoside antibiotics, glutathione, glycerol and glycerol derivatives, hypothalamus hormones, goserelin, grepafloxacin, gyrase inhibitors, guanethidine, gyrase inhibitors, haemin, halofantrine, haloperidol, urea derivatives as oral antidiabetics, heparin and heparin derivatives, cardiac glycosides, hyaluronic acid, hydralazine, hydrochlorothiazide and hydrochloro-thiazide derivatives, hydroxyomeprazole, hydroxyzine, ibritumomab, ibuprofen, idarubicin, ifliximab, ifosfamide, iloprost, imatinib, imidapril, imiglucerase, imipramine, imiquimod, imidapril, indometacin, indoramine, infliximab, insulin, insulin glargin, interferons, irbesartan, irinotecan, isoconazole, isoprenaline, itraconazole, ivabradines, fiodine and iodine derivatives, St. John's wort, potassium salts, ketoconazole, ketoprofen, ketotifen, lacidipine, lansoprazole, laronidase, latanoprost, leflunomide, lepirudin, lercanidipine, leteprinim, letrozole, levacetylmethadol, levetiracetam, levocetirizine, levodopa, levodrpropicin, levomethadone, licofelone, linezolide, lipinavir, lipoic acid and lipoic acid derivatives, lisinopril, lisuride, lofepramine, lodoxamide, lomefloxacin, lomustine, loperamide, lopinavir, loratadine, lornoxicam, losartan, lumefantrine, lutropine, magnesium salts, macrolide antibiotics, mangafodipir, maprotiline, mebendazole, mebeverine, meclozine, mefenamic acid, mefloquine, meloxicam, memantine, mepindolol, meprobamate, meropenem, mesalazine, mesuximide, metamizole, metformin, methadone, methotrexate, methyl 5-amino-4-oxopentanoate, methylnaloxone, methylnaloxone, methylnaltrexones, methylphenidate, methylprednisolone, metixen, metoclopramide, metoprolol, metronidazole, mianserin, mibefradil, miconazole, mifepristone, miglitol, miglustad, minocycline, minoxidil, misoprostol, mitomycin, mizolastine, modafinil, moexipril, montelukast, moroctocog, morphinans, morphine and morphine derivatives, moxifloxacin, ergot alkaloids, nalbuphine, naloxone, naproxen, naratriptan, narcotine, natamycin, nateglinide, nebivolol, nefazodone, nelfinavir, neostigmine, neramexan, nevirapine, nicergoline, nicethamide, nifedipine, niflumic acid, nimodipine, nimorazole, nimustine, nesiritide, nisoldipine, norfloxacin, novamine sulphone, noscapine, nystatin, ofloxacin, oktotride, olanzapine, olmesartan, olsalazine, oseltamivir, omeprazole, omoconazole, ondansetron, orlistat, oseltamivir, oxaceprol, oxacillin, oxaliplatin, oxaprozin, oxcarbacepin, oxicodone, oxiconazole, oxymetazoline, palivizumab, palanosetron, pantoprazole, paracetamol, parecoxib, paroxetine, pegaspargase, peginterferon, pegfilgrastrim, penciclovir, oral penicillins, pentazocine, pentifylline, pentoxifylline, peptide antibiotics, perindopril, perphenazine, pethidine, plant extracts, phenazone, pheniramine, phenylbutyric acid, phenytoin, phenothiazines, phenserine, phenylbutazone, phenytoin, pimecrolimus, pimozide, pindolol, pioglitazone, piperazine, piracetam, pirenzepine, piribedil, pirlindol, piroxicam, pramipexol, pramlintide, pravastatin, prazosin, procaine, promazine, propiverine, propranolol, propionic acid derivatives, propyphenazone, prostaglandins, protionamide, proxyphylline, quetiapine, quinapril, quinaprilate, quinupristine, ramipril, ranitidine, rabeprazole, raloxifen, ranolazine, rasburicase, reboxetin, repaclinides, reproterol, reserpine, revofloxacin, ribavirin, rifampicin, riluzoles, rimexolone, risedronate, risperidone, ritonavir, rituximab, rivastimen, risatriptan, rofecoxib, ropinirol, ropivacaine, rosiglitazone, roxatidine, roxithromycin, ruscogenin, rosuvastatin, rutoside and rutoside derivatives, sabadilla, salbutamol, salicylates, salmeterol, saperconazoles, thyroid hormones, scopolamine, selegiline, sertaconazole, sertindole, sertraline, sevelamer, sibutramine, sildenafil, silicates, simvastatin, sirolimus, sitosterol, sotalol, spaglumic acid, sparfloxacin, spectinomycin, spiramycin, spirapril, spironolactone, stavudine, streptomycin, sucralfate, sufentanil, sulbactam, sulphonamides, sulphasalazine, sulpiride, sultamicillin, sultiam, sumatriptan, suxamethonium chloride, tacrine, tacrolimus, tadalafil, taliolol, talsaclidine, tamoxifen, tasonermin, tazarotene, tegafur, tegaserod, telithromycin, telmisartan, temoporfin, temozolomide, tenatoprazole, tenecteplase, teniposide, tenofovir, tenoxicam, teriparatide, terazosin, terbinafine, terbutaline, terfenadine, teriparatide, terlipressin, tertatolol, testosterone and testosterone derivatives, tetracyclines, tetryzoline, tezosentan, theobromine, theophylline, theophylline derivatives, thiamazole, thiotepa, thr. growth factors, tiagabine, tiapride, tibolone, ticlopidine, tilidine, timolol, tinidazole, tioconazole, tioguanine, tiotropium, tioxolone, tirazetam, tiropramide, trofiban, tizanidine, tolazoline, tolbutamide, tolcapone, tolnaftate, tolperisone, tolterodine, topiramate, topotecan, torasemide, tramadol, tramazoline, trandolapril, tranylcypromine, trapidil, trastuzumab, travoprost, trazodone, trepostinil, triamcinolone and triamcinolone derivatives, triamterene, trifluperidol, trifluridine, trimetazidines, trimethoprim, trimipramine, tripelennamine, triprolidine, trifosfamide, tromantadine, trometamol, tropalpine, trovafloxacin, troxerutin, tulobuterol, trypsins, tyramine, tyrothricin, urapidil, ursodeoxycholic acid, theophylline ursodeoxycholic acid, valaciclovir, valdecoxib, valganciclovir, valproic acid, valsartan, vancomycin, vardenafil, vecuronium chloride, venlafaxine, verapamil, verteporfin, vidarabine, vigabatrine, viloxazine, vinblastine, vincamine, vincristine, vindesine, vinorelbine, vinpocetine, viquidil, vitamin D and derivatives of vitamin D, voriconazole, warfarin, xantinol nicotinate, ximelagatran, xipamide, zafirlukast, zalcitabine, zaleplon, zanamivir, zidovudine, ziprasidone, zoledronic acid, zolmitriptan, zolpidem, zoplicone, zotepine and the like.

Particularly Preferred Active Ingredients

Examples of particularly preferred active ingredients are metoprolol succinate and terbutaline sulphate.

The active ingredients can if desired also be used in the form of their pharmaceutically acceptable salts or derivatives, and in the case of chiral active ingredients it is possible to employ both optically active isomers and racemates or mixtures of diastereomers. If desired, the compositions of the invention may also comprise two or more active pharmaceutical ingredients.

The Outer Controlling Layer d)

The outer controlling layer d) comprises at least 60, preferably at least 80, particularly preferably 90 to 100, % by weight of one or a mixture of a plurality of (meth)acrylate copolymers composed of 98 to 85 C₁ to C₄ alkyl esters of (meth)acrylic acid and 2 to 15% by weight of methacrylate monomers with a quaternary ammonium group in the alkyl radical, and, where appropriate, up to 40, preferably up to 20, in particular 0 to 10, % by weight of further pharmaceutically usable polymers. However, is particularly preferred for no further pharmaceutically usable polymers to be present. The data on the % by weight of the abovementioned polymers in the outer controlling layer d) are moreover calculated without taking account of any pharmaceutically usual excipients which are additionally present.

Appropriate (meth)acrylate copolymers are disclosed for example in EP-A 181 515 or DE patent 1 617 751. They are polymers which are soluble or swellable irrespective of the pH and are suitable for medicament coatings. A possible production process to be mentioned is bulk polymerization in the presence of an initiator which forms free radicals and is dissolved in the monomer mixture. The polymer can likewise be produced by means of solution or precipitation polymerization. The polymer can be obtained in this way in the form of a fine powder, achievable in the case of bulk polymerization by grinding and in the case of solution and precipitation polymerization for example by spray drying.

The (meth)acrylate copolymer is composed of 85 to 98% by weight of free-radical polymerized C₁ to C₄ alkyl esters of acrylic or methacrylic acid and 15 to 2% by weight of (meth)acrylate monomers with a quaternary ammonium group in the alkyl radical.

Preferred C₁ to C₄ alkyl esters of acrylic or methacrylic acid are methyl acrylate, ethyl acrylate, butyl acrylate, butyl methacrylate and methyl methacrylate.

The particularly preferred (meth)acrylate monomer with quaternary ammonium groups is 2-trimethylammoniumethyl methacrylate chloride.

An appropriate copolymer may be composed for example of 50-70% by weight of methyl methacrylate, 20-40% by weight of ethyl acrylate and 7-2% by weight of 2-trimethylammoniumethyl methacrylate chloride.

A specifically suitable copolymer comprises 65% by weight of methyl methacrylate, 30% by weight of ethyl acrylate and 5% by weight of 2-trimethylammoniumethyl methacrylate chloride be composed (EUDRAGIT® RS).

A further suitable (meth)acrylate copolymer may be composed for example of 85 to less than 93% by weight of C₁ to C₄ alkyl esters of acrylic or methacrylic acid and more than 7 to 15% by weight of (meth)acrylate monomers with a quaternary ammonium group in the alkyl radical. Such (meth)acrylate monomers are commercially available and have long been used for release-slowing coatings.

A specifically suitable copolymer comprises for example 60% by weight of methyl methacrylate, 30% by weight of ethyl acrylate and 10% by weight of 2-trimethyl-ammoniumethyl methacrylate chloride (EUDRAGIT® RL).

It is possible where appropriate for up to 40, preferably up to 20, in particular 0 to 10, % by weight of further pharmaceutically usable polymers to be present in the outer controlling layer d). Examples of suitable polymers are:

copolymers of methyl methacrylate and/or ethyl acrylate and methacrylic acid, copolymers of methyl methacrylate, methyl acrylate and methacrylic acid, copolymers of methyl methacrylate, butyl methacrylate and dimethylethyl methacrylate, copolymers of methyl methacrylate, ethyl acrylate and trimethylammoniumethyl methacrylate, copolymers of methyl methacrylate and ethyl acrylate, copolymers of ethyl acrylate, methyl acrylate, butyl methacrylate and methacrylic acid,

polyvinylpyrolidones (PVPs), polyvinyl alcohols, polyvinyl alcohol-polyethylene glycol graft copolymer (Kollicoat®), starch and derivatives thereof, polyvinyl acetate phthalate (PVAP, Coateric®), polyvinyl acetate (PVAc, Kollicoat), vinyl acetate/vinylpyrolidone copolymer (Kollidone® VA64), vinyl acetate: crotonic acid 9:1 copolymer (VAC: CRA, Kollicoat® VAC), polyethylene glycols with a molecular weight above 1000 (g/mol), chitosan, a (meth)acrylate copolymer consisting of 20-40% by weight of methyl methacrylate and 60 to 80% by weight of methacrylic acid, a crosslinked and/or uncrosslinked polyacrylic acid, an Na alginate, and/or a pectin,

celluloses such as, for example, anionic carboxymethyl-cellulose and salts thereof (CMC, Na—CMC, Ca—CMC, Blanose, Tylopur), carboxymethylethylcellulose (CMEC, Duodcell®), hydroxyethylcellulose (HEC, Klucel), hydroxypropylcellulose (HPC), hydroxypropylmethyl-cellulose (HPMC, Pharmacoat, Methocel, Sepifilm, Viscontran, Opadry), hydroxymethylethylcellulose (HEMC), ethylcellulose (EC, Ethocel®, Aquacoat®, Surelease®), methylcellulose (MC, Viscontran, Tylopur, Methocel), cellulose esters, cellulose glycolate, cellulose acetate phthalate (CAP, Cellulosi acetas, PhEur, cellulose acetate phthalate, NF, Aquateric®), cellulose acetate succinate (CAS), cellulose acetate trimeliate (CAT), hydroxypropylmethylcellulose phthalate (HPMCP, HP50, HP55), hydroxypropylmethylcellulose acetate succinate (HPMCAS-LF, -MF, -HF).

Layer Thicknesses and Proportions by Weight

Core Layer a)

The core layer a) (without nonpareilles) may have an average diameter in the range from about 100 to 800, preferably 250 to 500 μm (corresponding to a range from about 60 to 40 mesh).

Inner Controlling Layer b)

The inner controlling layer b) may have a proportion by weight of from 0.5 to 80, preferably 2.5 to 50, particularly preferably 5 to 40, % by weight based on the core layer a). It is favourable for the layer thickness to be about 1 to 100, preferably 5 to 50, in particular 10 to 40, μm.

Active Ingredient Layer c)

The active ingredient layer c) may account for 10 to 400, preferably 50 to 200, % by weight based on the core layer a) and the inner controlling layer b).

Outer Controlling Layer d)

The outer controlling layer d) may have a proportion by weight of from 2.5 to 100, preferably 10 to 70, particularly preferably 20 to 60, % by weight based on the core layer a), the inner controlling layer b) and the active ingredient layer c). The layer thickness is about 4 to 150, in particular 15 to 75, particularly preferably 30 to 70, μm.

Excipients Customary in Pharmacy

Layers a), b), c) and d) may additionally and in a manner known per se comprise excipients customary in pharmacy.

Excipients customary in pharmacy, occasionally also referred to as customary additives, are added to the formulation of the invention, preferably during production of the granules or powders. It is, of course, always necessary for all the substances employed to be toxicologically acceptable and usable in particular in medicaments without a risk for patients.

The amounts employed and the use of excipients customary in pharmacy for medicament coatings or layerings are familiar to the skilled worker. Examples of possible excipients or additives customary in pharmacy are release agents, pigments, stabilizers, antioxidants, pore formers, penetration promoters, gloss agents, aromatizing substances or flavourings. They serve as processing aids and are intended to ensure a reliable and reproducible production process and good long-term storage stability or they achieve additional advantageous properties in the pharmaceutical form. They are added to the polymer preparations before processing and may influence the permeability of the coatings, it being possible to utilize this where appropriate as additional control parameter.

Release Agents:

Release agents usually have lipophilic properties and are usually added to the spray suspensions. They prevent agglomeration of the cores during the film coating. Talc, Mg stearate or Ca stearate, ground silica, kaolin or nonionic emulsifiers with an HLB of between 3 and 8 are preferably employed. The usual amounts employed of release agent are between 0.5 to 100% by weight based on the weight of the cores.

Pigments:

Pigments incompatible with the coating agent are in particular those pigments which, if added directly to the (meth)acrylate copolymer dispersion, e.g. by stirring in, in the usual amounts used of, for example, 20 to 400% by weight based on the dry weight of the (meth)acrylate copolymer, lead to destabilization of the dispersion, coagulation, to signs of inhomogeneity or similarly unwanted effects. The pigments to be used are moreover of course non-toxic and suitable for pharmaceutical purposes. Concerning this, see also, for example: Deutsche Forschungsgemeinschaft, Farbstoffe für Lebensmittel, Harald, Boldt Verlag KG, Boppard (1978); Deutsche Lebensmittelrundschau 74, No. 4, p. 156 (1978); Arzneimittelfarbstoffverordnung AmFarbV of 25.08.1980.

Pigments incompatible with the coating agent may be for example alumina pigments. Examples of incompatible pigments are orange yellow, cochineal red lake, coloured pigments based on alumina or azo dyes, sulphonic acid dyes, orange yellow S (E110, C.I. 15985, FD&C Yellow 6), indigo carmine (E132, C.I. 73015, FD&C Blue 2), tartrazine (E 102, C.I. 19140, FD&C Yellow 5), Ponceau 4R (E 125, C.I. 16255, FD&C Cochineal Red A), quinoline yellow (E 104, C.I. 47005, FD&C Yellow 10), erythrosine (E127, C.I. 45430, FD&C Red 3), azorubine (E 122, C.I. 14720, FD&C Carmoisine), amaranth (E 123, C.I. 16185, FD&C Red 2), acid brilliant green (E 142, C.I. 44090, FD&C Green S).

The E numbers indicated for the pigments relate to an EU numbering. Concerning this, see also “Deutsche Forschungsgemeinschaft, Farbstoffe für Lebensmittel, Harald Boldt Verlag KG, Boppard (1978); Deutsche Lebensmittelrundschau 74, No. 4, p. 156 (1978); Arzneimittelfarbstoffverordnung AmFarbV of 25.08.1980. The FD&C numbers relate to the approval in food, drugs and cosmetics by the U.S. food and drug administration (FDA) described in: U.S. Food and Drug Administration, Center for Food Safety and Applied Nutrition, Office of Cosmetics and Colors: Code of Federal Regulations—Title 21 Color Additive Regulations Part 82, Listing of Certified Provisionally Listed Colors and Specifications (CFR 21 Part 82).

Plasticizers

Further additives may also be plasticizers. The usual amounts are between 0 and 50, preferably 5 to 20, % by weight based for example on the (meth)acrylate copolymer of the outer layer d).

Plasticizers may influence the functionality of the polymer layer, depending on the type (lipophilic or hydrophilic) and added amount. Plasticizers achieve through physical interaction with the polymers a reduction in the glass transition temperature and promote film formation, depending on the added amount. Suitable substances usually have a molecular weight of between 100 and 20 000 and comprise one or more hydrophilic groups in the molecule, e.g. hydroxyl, ester or amino groups.

Examples of suitable plasticizers are alkyl citrates, glycerol esters, alkyl phthalates, alkyl sebacates, sucrose esters, sorbitan esters, diethyl sebacate, dibutyl sebacate and polyethylene glycols 200 to 12 000. Preferred plasticizers are triethyl citrate (TEC), acetyl triethyl citrate (ATEC) and dibutyl sebacate (DBS). Mention should additionally be made of esters which are usually liquid at room temperature, such as citrates, phthalates, sebacates or castor oil. Esters of citric acid and sebacic acid are preferably used.

Addition of the plasticizers to the formulation can be carried out in a known manner, directly, in aqueous solution or after thermal pretreatment of the mixture. It is also possible to employ mixtures of plasticizers.

Processes for Producing a Multilayer Pharmaceutical Form

The multilayer pharmaceutical form can be produced in a manner known per se by means of usual pharmaceutical processes such as direct compression, compression of dry, wet or sintered granules, extrusion and subsequent rounding off, wet or dry granulation or direct pelleting (e.g. on plates) or by binding of powders (powder layering) onto active ingredient-free beads or cores (nonpareilles) or active ingredient-containing particles, by means of spray processes or fluidized bed granulation. Application of the inner and outer controlling layers b) and c) can take place by means of known and usual processes such as, for example, spray application of polymer solutions or polymer dispersions.

Examples of Standard Process Parameters

The following standard process parameters are intended to explain examples of possible procedures in the production process.

Stage 1: (Formulation of a Core Layer a))

Crystal cores in the range of 400 μm-800 μm are selected for the experiments.

Stage 2: (Application of an Inner Controlling Layer b))

Modulating layer with EUDRAGIT® NE (copolymer of 50% by weight of methyl methacrylate and 50% by weight of ethyl acrylate)

20% w/w EUDRAGIT® NE 30 D suspension is used as the basic modulating layer for most experiments. The formulation comprises 15% solids in dispersion with 20% polymer, 5% glycerol monostearate (GMS-900), 2% Tween 80 and 0.5% of a pigment.

This layer is applied to the crystal cores using a fluidized bed apparatus.

Process parameters:

| | |
|---|---|
| Inlet air temperature: | 32° C. |
| Product temperature: | 30° C. |
| Outlet air temperature: | 23° C. |
| Pump rpm: | 8-10 (5-10 g/min) |
| Processing time: | 120-160 min |
| Drying process: | 2 hours in convection oven at 40° C. |

Stage 3 (Application of an Active Ingredient Layer c))

The active ingredient can be applied to simple crystal cores or to crystal cores coated with a substance having a modulating effect, until a weight gain of 100 to 200% is obtained. Active ingredient application can also be carried out with additional salt integration in order to increase the salt concentration in the pellets. Active ingredient application is carried out for example in a coating pan using the known “powder layering” process.

General Process Parameters for the Active Ingredient Application

| | |
|---|---|
| Spraying time | 90 min |
| Total volume | 543 g |
| Weight/powder in portions | 15 g |
| Nozzle | 1.00 mm |
| Spraying pressure | low |
| Coating pan speed | 24-25 rpm |
| Pumping speed | 12 rpm (9 g/min) |
| Drying in the apparatus | 5 min |
| Final drying in a convection oven | 12 h at 40° C. |
| Outlet air conditions | on |

The active ingredient-coated pellets obtained in this way may be in the size range of 600-1200 μm and be used for further coating with EUDRAGIT® RS (copolymer of 65% by weight of methyl methacrylate, 30% by weight of ethyl acrylate and 5% by weight of 2-trimethylammoniumethyl methacrylate chloride).

Stage 4 (Application of an Outer Controlling Layer d) Consisting of a Release-Slowing Coating with (EUDRAGIT® RS)

The active ingredient-coated pellets can be coated for example with EUDRAGIT® RS, applying various amounts (from 10-50%) in a fluidized bed apparatus. A formulation may comprise for example: 20% solids in EUDRAGIT® RS dispersion with 50% talc, 20% triethyl citrate, 0.5% pigments.

Process parameters:

| | |
|---|---|
| Inlet air temperature: | 35° C. |
| Product temperature: | 32° C. |
| Outlet air temperature | 24° C. |
| Pump rpm: | 8-16 (4-8 g/min) |
| Processing time: | 120-180 min |
| Drying process: | 2 h in a convection oven at |
| | 40° C. |

### SPECIFIC EXAMPLES

### Example I

Modulated layer concentration up to 10% w/w:

Trisodium citrate crystals were coated with 10% w/w EUDRAGIT® NE 30D. Theophylline is applied to this layer until the weight gain is 200%. These coated cores are further coated with 20-40% w/w EUDRAGIT® RS30D.

### Example II

Modulated layer concentration up to 20% w/w:

Trisodium citrate crystals are coated with 20% w/w EUDRAGIT® NE 30D. Theophylline is applied to this layer until the weight gain is 200%. These coated cores are further coated with 20-40% w/w EUDRAGIT® RS30D.

### Example III

Increasing the salt concentration in the finished pellet:

Sodium chloride cores were first coated with a modulating layer of EUDRAGIT® NE 30D up to 20% w/w. Theophylline and ground sodium chloride crystals were applied to this layer until the weight gain was 200%. These coated pellets were further coated with 20-40% w/w EUDRAGIT® RS30D.

### Example IV

Effect of various salts:

Sodium chloride and sodium acetate crystals are first coated with EUDRAGIT® NE 30 D up to 20% w/w. Theophylline is applied to this layer until the weight gain is 200%. These coated pellets are further coated with 20-40% w/w EUDRAGIT® RS30D.

Possible Release Characteristics

The multilayer pharmaceutical form is particularly suitable for achieving specific active ingredient release characteristics. Mention should be made of active ingredient release characteristics of zero order (linear), 1st order (accelerated), fast-slow, slow-fast release characteristics.

Pharmaceutical Form for the Active Ingredient Metoprolol Succinate

The active ingredient metoprolol succinate which can be employed for the therapy of hypertension and angina is advantageously formulated in a pharmaceutical form which can be taken before going to bed, initially releases the active ingredient in linear fashion but changes after 4 to 6 hours to an accelerated active ingredient delivery. It is thus possible to counter the risk of high blood pressure and myocardial infarctions which is particularly high in the early morning.

Four possible variants which with which the desired release characteristics for the active ingredient metoprolol succinate can be achieved are disclosed according to the invention.

| | Example M1 | Example M2 | Example M3 | Example M4 |
|---|---|---|---|---|
| Core layer a) | Na acetate crystals | NaCl crystals | NaCl crystals | NaCl crystals |
| Inner controlling layer b) | 20 wt % | 20 wt % | 40 wt % | 20 wt % |
| [wt % based on a)] | EUDRAGIT ® NE | EUDRAGIT ® NE | EUDRAGIT ® NE | EUDRAGIT ® NE |
| Active ingredient layer c) | 200 wt % metoprolol | 200 wt % metoprolol | 200 wt % metoprolol | 200 wt % metoprolol |
| [wt % based on a) + b)] | succinate | succinate | succinate | succinate + NaCl |
| Outer controlling layer d) | 40 wt % | 50 wt % | 50 wt % | 50 wt % |
| [wt % based on a), b) + c)] | EUDRAGIT ® RS | EUDRAGIT ® RS | EUDRAGIT ® RS | EUDRAGIT ® RS |

EUDRAGIT ® RS = copolymer of 65% by weight methyl methacrylate, 30% by weight ethyl acrylate and 5% by weight 2-trimethylammoniumethyl methacrylate chloride.
EUDRAGIT ® NE = copolymer of 50% by weight methyl methacrylate and 50% by weight ethyl acrylate.

The release characteristics of the pellets from Example M4 were tested in the USP <711> dissolution test, apparatus 1, phosphate buffer of pH 6.8. It was found in this case that about 11% of the contained active ingredient was released in each case up to the second and from the second to the fourth hour. There was observed to be an accelerated active ingredient delivery of about 15% from the fourth hour to the sixth hour and of 20% in each case from the sixth to the eighth and the eighth to the tenth hour. Active ingredient delivery slowed again from the tenth hour onwards.

Metoprolol succinate release of the pellets
from Example M4 (USP I, 100 rpm, pH 6.8)

| | Active ingredient delivery | Cumulative active |
|---|---|---|
| Hour | in the 2-hour interval | ingredient delivery |
| 2 | 11 | 11 |
| 4 | 11 | 22 |
| 6 | 15 | 37 |
| 8 | 20 | 57 |
| 10 | 20 | 77 |
| 12 | 11 | 88 |

Pharmaceutical Form for the Active Ingredient Terbutaline Sulphate

The active ingredient terbutaline sulphate is a beta 2 agonist which can be employed for the therapy of asthma. A formulation with approximately constant rate of active ingredient delivery is prepared according to the invention. Acute asthma symptoms can are alleviated thereby immediately after intake of the pharmaceutical form. Thereafter, uniform amounts of the active ingredient are delivered to suppress the flaring up again of further symptoms. It is therefore unnecessary for single doses to be administered several times a day, repeatedly and more or less punctually, as is the case with most prior art pharmaceutical forms. This is overall more convenient, more acceptable (patient compliancy) and in many cases also more tolerable for the patient.

Two possible variants which with which the desired release characteristics for the active ingredient terbutaline sulphate can be achieved are disclosed according to the invention.

| | Example T1 | Example T2 |
|---|---|---|
| Core layer a) | Na acetate | NaCl crystals |
| | crystals | |
| Inner controlling | 20 wt % | 20 wt % |
| layer b) | EUDRAGIT ® | EUDRAGIT ® |
| [wt % based on a)] | NE | NE |
| Active ingredient layer c) | 200 wt % | 200 wt % |
| [wt % based on a) + b)] | terbutaline | terbutaline |
| | sulphate | sulphate + |
| | | NaCl |
| Outer controlling | 30% wt % | 30% wt % |
| layer d) | EUDRAGIT ® | EUDRAGIT ® |
| [wt % based on a), | RS | RS |
| b) + c)] | | |

EUDRAGIT ® RS = copolymer of 65% by weight methyl methacrylate, 30% by weight ethyl acrylate and 5% by weight 2-trimethylammoniumethyl methacrylate chloride.
EUDRAGIT ® NE = copolymer of 50% by weight methyl methacrylate and 50% by weight ethyl acrylate.

The release characteristics of the pellets from Example M4 were tested in the USP <711> dissolution test, apparatus 1, phosphate buffer of pH 6.8. It was found in this case that approximately constant amounts of active ingredient are released in 2-hour intervals.

Terbutaline sulphate release of the pellets
from Example T2 (USP I, 100 rpm, pH 6.8)

| | Active ingredient delivery | Cumulative % active |
|---|---|---|
| Hour | in the 2-hour interval | ingredient delivery |
| 2 | 14 | 14 |
| 4 | 17 | 31 |
| 6 | 14 | 45 |
| 8 | 10 | 55 |
| 10 | 9 | 64 |
| 12 | 10 | 74 |

Dosage Forms/Uses

The multilayer pharmaceutical forms of the invention are initially in the form of tablets or pellets. These can in turn be used as ingredient of a multiparticulate pharmaceutical form, of pellet-containing tablets, minitablets, capsules, sachets, effervescent tablets or powders for reconstitution. It is possible according to the invention for multiparticulate pharmaceutical forms also to include in particular mixtures of formulated pellets comprising different active ingredients. A further possibility is for multiparticulate pharmaceutical forms of the invention to comprise pellet populations which are loaded with one and the same active ingredient but are differently formulated e and show different release profiles. It is possible in this way for mixed release profiles of one or more active ingredients to be achieved and for a more refined adaptation for the desired therapy to be carried out via the mixtures.

### Examples

EUDRAGIT® RS=copolymer of 65% by weight of methyl methacrylate, 30% by weight of ethyl acrylate and 5% by weight of 2-trimethylammoniumethyl methacrylate chloride.

EUDRAGIT® NE=copolymer of 50% by weight of methyl methacrylate and 50% by weight of ethyl acrylate.

### Examples 1-5

### Not According to the Invention

In order to examine the influence of various substances having a modulating effect on the outer controlling layer d), pellets without an inner controlling layer b) were produced. Pellets without a substance having a modulating effect but with microcrystalline cellulose (Example 5) were used for comparison. It is possible in this way to ascertain effects such as an accelerated or a slowed active ingredient delivery irrespective of an inner controlling layer.

A mixture of 1290 g of theophylline powder, 65 g of Kollidon 25 and 6.5 g of Aerosil 200 are sprinkled onto 700 g of core material in a coating pan and bound to the core material by simultaneous spraying of a solution of 33 g of theophylline and 10 of Kollidon 25 in 500 g of demineralized water. A spray suspension of 400 g of EUDRAGIT® RS 30 D (corresponding to 120 g of polymer), 60 g of talc, 24 g of triethyl citrate, 0.6 g of yellow iron oxide and 538.3 g of demineralized water is applied in a fluidized bed system to 600 g of the theophylline pellets produced in this way with non-slow-release modulator core. The applied amount of polymer thus corresponds to 20% of the starting material.

The pellets produced in Example 1-5 were investigated for active ingredient delivery in a PhEur phosphate buffer of pH 6.8 in a USP dissolution tester:

| | Example | | | | |
|---|---|---|---|---|---|
| | 1 | 2 | 3 | 4 | 5 |
| Core layer a) | Sodium acetate | Sodium chloride | Sodium succinate | Citric acid | Microcrystalline |
| | crystals | crystals | crystals | crystals | cellulose granules |
| Inner controlling | — | — | — | — | — |
| layer b) | | | | | |
| Active ingredient | theophylline | theophylline | theophylline | theophylline | theophylline |
| layer c) | | | | | |
| Outer controlling | EUDRAGIT ® | EUDRAGIT ® | EUDRAGIT ® | EUDRAGIT ® | EUDRAGIT ® |
| layer d) | RS 30 D | RS 30 D | RS 30 D | RS 30 D | RS 30 D |
| Time [h] | | | | | |
| 0 | 0 | 0 | 0 | 0 | 0 |
| 0.5 | 3.1 | 0.4 | 7.0 | 6.3 | 1.8 |
| 1 | 5.4 | 1.1 | 13.2 | 10.2 | 3.0 |
| 2 | 9.2 | 2.1 | 28.2 | 18.1 | 5.2 |
| 4 | 14.8 | 3.9 | 65.9 | 35.1 | 11.6 |
| 6 | 20.1 | 5.5 | 77.9 | 51.0 | 20.7 |
| 8 | 25.0 | 7.1 | 89.7 | 66.8 | 30.9 |
| 10 | 29.1 | 8.4 | 96.3 | 80.0 | 42.7 |

The release values show the first order profile characteristic of diffusion processes. Thus, without control of modulator release, an equilibrium very quickly results in the coated pellet, which definitively adjusts the permeability of the final coating at the start of release.

The release profile of the pellets with microcrystalline cellulose (Example 5) is between those with sodium acetate and sodium chloride. Thus, an accelerating effect results for sodium acetate, citric acid and sodium succinate, and a reducing effect results for sodium chloride.

### Examples 6-10

(According to the invention, “linearly” zero order release characteristics).

1000 g of core material are coated in a fluidized bed system with a spray suspension of 666 g of EUDRAGIT NE 30 D (corresponding to 200 g of polymer), 4 g of polysorbate 80, 10 g of glycerol monostearate, 1 g of yellow iron oxide and 720 g of demineralized water. The applied amount of polymer thus corresponds to 20% of the starting material.

A mixture of 1290 g of theophylline powder, 65 g of Kollidon 25 and 6.5 g of Aerosil 200 are sprinkled onto 700 g of the cores produced in this way with slow-release modulator delivery in a coating pan and bound to the core material by simultaneous spraying of a solution of 33 g of theophylline and 10 of Kollidon 25 in 500 g of demineralized water.

A spray suspension of 400 g of EUDRAGIT® RS 30 D (corresponding to 120 g of polymer), 60 g of talc, 24 g of triethyl citrate, 0.6 g of yellow iron oxide and 538.3 g of demineralized water is applied to 600 g of the theophylline pellets produced in this way with slow-release modulator core in a fluidized bed system.

The applied amount of polymer thus corresponded to 20% of the starting material.

The pellets produced in Example 6-10 were investigated for active ingredient delivery in a PhEur phosphate buffer of pH 6.8 in a USP dissolution tester:

| | Example | | | | |
|---|---|---|---|---|---|
| | 6 | 7 | 8 | 9 | 10 |
| Core layer a) | Sodium acetate | Sodium chloride | Sodium citrate | Sodium succinate | Citric acid |
| | crystals | crystals | crystals | crystals | crystals |
| Inner controlling | EUDRAGIT ® | EUDRAGIT ® | EUDRAGIT ® | EUDRAGIT ® | EUDRAGIT ® |
| layer b) | NE 30 D | NE 30 D | NE 30 D | NE 30 D | NE 30 D |
| Active ingredient | theophylline | theophylline | theophylline | theophylline | theophylline |
| layer c) | | | | | |
| Outer controlling | EUDRAGIT ® | EUDRAGIT ® | EUDRAGIT ® | EUDRAGIT ® | EUDRAGIT ® |
| layer d) | RS 30 D | RS 30 D | RS 30 D | RS 30 D | RS 30 D |
| Time [h] | Active ingredient delivery [%] | | | | |
| 0 | 0 | 0 | 0 | 0 | 0 |
| 0.5 | 1.7 | 3.2 | 6.7 | 11.6 | 29.3 |
| 1 | 3.1 | 6.3 | 16.4 | 21.9 | 57.7 |
| 2 | 6.4 | 14.5 | 39.2 | 75.9 | 87.9 |
| 4 | 16.1 | 27.5 | 75.4 | 99.0 | 94.3 |
| 6 | 23.2 | 40.0 | 90.4 | | |
| 8 | 29.9 | 48.6 | | | |
| 10 | 38.2 | 63.6 | | | |

The release values show a zero order profile, i.e. they are virtually linear. The modulator release from the core layer a) thus prevents early active ingredient delivery from the system in the case of sodium succinate and citric acid, and thus the accelerating effect is retained over a longer period. In the case of sodium citrate and sodium acetate, the highest possible increase in permeability of the EUDRAGIT® RS coating is never reached through delaying the modulator supply, and therefore a continuous resupply results in a longer and linear release plot compared with the uncontrolled modulator from Example 1 and 3. In the case of the sodium chloride core, reducing effect is retained longer through a continuous resupply, thus achieving a slower linear release.

### Example 11

### Not According to the Invention

To examine the theory that the control possibilities found require the use of an ionic coating material, pellets with a neutral coating material were investigated in the following examples:

A mixture of 1290 g of theophylline powder, 65 g of Kollidon 25 and 6.5 g of Aerosil 200 are sprinkled onto 700 g of sodium acetate crystals in a coating pan and bound to the core material by simultaneous spraying of a solution of 33 g of theophylline and 10 of Kollidon 25 in 500 g of demineralized water. A spray suspension of 400 g of EUDRAGIT® NE 30 D (corresponding to 120 g of polymer), 2.4 g of polysorbate 80, 6 g of glycerol monostearate, 0.6 g of yellow iron oxide and 432 g of demineralized water was applied to 600 g of theophylline pellets produced in this way with a non-slow-release modulator core in a fluidized bed system.

### Example 12

### Not according to the Invention

A mixture of 1290 g of theophylline powder, 65 g of Kollidon 25 and 6.5 g of Aerosil 200 are sprinkled onto 700 g of sodium chloride crystals in a coating pan and bound to the core material by simultaneous spraying of a solution of 33 g of theophylline and 10 of Kollidon 25 in 500 g of demineralized water. A spray suspension of 400 g of EUDRAGIT® NE 30 D (corresponding to 120 g of polymer), 2.4 g of polysorbate 80, 6 g of glycerol monostearate, 0.6 g of yellow iron oxide and 432 g of demineralized water was applied to 600 g of theophylline pellets produced in this way with a non-slow-release modulator core in a fluidized bed system.

| | Example | | | |
|---|---|---|---|---|
| | 1 | 6 | 11 | 12 |
| Core layer a) | Sodium acetate | Sodium acetate | Sodium acetate | Sodium acetate |
| | crystals | crystals | crystals | crystals |
| Inner controlling | — | EUDRAGIT ® | — | EUDRAGIT ® |
| layer b) | | NE 30 D | | NE 30 D |
| Active ingredient | theophylline | theophylline | theophylline | theophylline |
| layer c) | | | | |
| Outer controlling | EUDRAGIT ® | EUDRAGIT ® | EUDRAGIT ® | EUDRAGIT ® |
| layer d) | RS 30 D | RS 30 D | NE 30 D | NE 30 D |

| | |
|---|---|
| Time [h] | Active ingredient delivery [%] |

| | | | | |
|---|---|---|---|---|
| 0 | 0 | 0 | 0 | 0 |
| 0.5 | 3.1 | 1.7 | 8.96 | 6.74 |
| 1 | 5.4 | 3.1 | 14.66 | 11.56 |
| 2 | 9.2 | 6.4 | 22.61 | 18.67 |
| 4 | 14.8 | 16.1 | 38.33 | 32.11 |
| 6 | 20.1 | 23.2 | 58.51 | 48.90 |
| 8 | 25.0 | 29.9 | 73.78 | 66.01 |
| 10 | 29.1 | 38.2 | 82.35 | 75.74 |

The effect of the inner controlling layer b) is evident on comparison of Example 1 with 6.
The effect of the outer controlling layer d) of the invention in Example 1 is evident on comparison of Example 1 with 11.
The effect of the absence of an outer controlling layer d) of the invention, irrespective of the presence of an inner controlling layer b), is evident on comparison of Example 11 with 12.

### Example 13

### Accelerated

1000 g of sodium acetate crystals are coated in a fluidized bed system with a spray suspension of 666 g of EUDRAGIT® NE 30 D (corresponding to 200 g of polymer), 4 g of polysorbate 80, 10 g of glycerol monostearate, 1 g of yellow iron oxide and 720 g of demineralized water. The applied amount of polymer thus corresponded to 20% of the starting material. A mixture of 760 g of theophylline powder, 560 g of sodium chloride, 65 g of Kollidon 25 and 6.5 g of Aerosil 200 were sprinkled onto 700 g of the cores produced in this way with slow-release modulator delivery in a coating pan and bound to the core material by simultaneous spraying of a solution of 10 of Kollidon 25 in 500 g of demineralized water. A spray suspension of 400 g of EUDRAGIT® RS 30 D (corresponding to 120 g of polymer), 60 g of talc, 24 g of triethyl citrate, 0.6 g of yellow iron oxide and 538.3 g of demineralized water is applied to 600 g of the theophylline pellets produced in this way with slow-release modulator in the core layer a) in a fluidized bed system. The applied amount of polymer thus corresponds to 20% of the starting material.

The pellets produced in Example 13 can be investigated for active ingredient delivery in a PhEur phosphate buffer of pH 6.8 in a USP dissolution tester. The following slow-release principle will be able to be ascertained in this way:

The active ingredient is released within a period of 10 hours, with the initial release being very small. A continuous acceleration of release is to be observed over the investigated period.

1. A multilayer pharmaceutical form for controlled active ingredient release, comprising
a) a core layer comprising a substance having a modulating effect in relation to active ingredient delivery, where appropriate a core and/or an active ingredient,
b) an inner controlling layer which influences the delivery of the substance having a modulating effect and of the active ingredient which is present where appropriate from the core layer, consisting of pharmaceutically usable polymers, waxes, resins and/or proteins,
c) an active ingredient layer comprising an active pharmaceutical ingredient and, optionally, a substance having a modulating effect,
d) an outer controlling layer comprising at least 60% by weight of one or a mixture of a plurality of (meth)acrylate copolymers composed of 98 to 85 C₁ to C₄ alkyl esters of (meth)acrylic acid and 2 to 15% by weight of methacrylate monomers with a quaternary ammonium group in the alkyl radical, and, optionally, up to 40% by weight of further pharmaceutically usable polymers,
where the layers may additionally comprise pharmaceutically acceptable excipients.

2. The multilayer pharmaceutical form according to claim 1, wherein the core layer a) alternatively and essentially comprises the following ingredients:
I. a substance having a modulating effect, e.g. in crystalline, granular or coprecipitate form,
II. a substance having a modulating effect and an active ingredient, which may be present in successive layers in any sequence or in a mixture,
III. a neutral core (nonpareilles) coated with a substance having a modulating effect,
IV. a neutral core (nonpareilles) coated with a substance having a modulating effect and with an active ingredient, which may be present in successive layers in any sequence or in a mixture.

3. The multilayer pharmaceutical form according to claim 1, wherein the inner controlling layer consists of a polymer which is insoluble in water or only swellable in water.

4. The multilayer pharmaceutical form according to claim 3, wherein the polymer is at least one selected from the group consisting of:
copolymers of methyl methacrylate and/or ethyl acrylate and methacrylic acid, copolymers of methyl methacrylate, methyl acrylate and methacrylic acid, copolymers of methyl methacrylate, butyl methacrylate and dimethylethyl methacrylate, copolymers of methyl methacrylate, ethyl acrylate and trimethylammoniumethyl methacrylate, copolymers of methyl methacrylate and ethyl acrylate, copolymers of ethyl acrylate, methyl acrylate, butyl methacrylate and methacrylic acid,
polyvinylpyrolidones (PVPs), polyvinyl alcohols, polyvinyl alcohol-polyethylene glycol graft copolymer (Kollicoat®), starch and derivatives thereof, polyvinyl acetate phthalate (PVAP, Coateric®), polyvinyl acetate (PVAc, Kollicoat), vinyl acetate/vinylpyrolidone copolymer (Kollidon® VA64), vinyl acetate: crotonic acid 9:1 copolymer (VAC: CRA, Kollicoat® VAC), polyethylene glycols with a molecular weight above 1000 (g/mol), chitosan, a (meth)acrylate copolymer consisting of 20 40% by weight of methyl methacrylate and 60 to 80% by weight of methacrylic acid, a crosslinked and/or uncrosslinked polyacrylic acid, an Na alginate, a pectin,
cellulose, anionic carboxymethylcellulose and salts thereof (CMC, Na—CMC, Ca—CMC, Blanose, Tylopur), carboxymethylethylcellulose (CMEC, Duodcell®), hydroxyethylcellulose (HEC, Klucel), hydroxypropylcellulose (HPC), hydroxypropylmethylcellulose (HPMC, Pharmacoat, Methocel, Sepifilm, Viscontran, Opadry), hydroxymethylethylcellulose (HEMC), ethylcellulose (EC, Ethocel®, Aquacoat®, Surelease®), methylcellulose (MC, Viscontran, Tylopur, Methocel), cellulose esters, cellulose glycolate, cellulose acetate phthalate (CAP, Cellulosi acetas, PhEur, cellulose acetate phthalate, NF, Aquateric®), cellulose acetate succinate (CAS), cellulose acetate trimeliate (CAT), hydroxypropylmethylcellulose phthalate (HPMCP, HP50, HP55), and hydroxypropylmethylcellulose acetate succinate (HPMCAS-LF, -MF, -HF).

5. The multilayer pharmaceutical form according to claim 1, wherein the inner controlling layer consists of a wax carnauba wax and/or beeswax.

6. The multilayer pharmaceutical form according to claim 1, wherein the matrix of the inner controlling layer comprises the resin shellac.

7. The multilayer pharmaceutical form according to claim 1, wherein the inner controlling layer consists of a protein such as, for example, albumin, gelatin, gluten, collagen and/or zein.

8. The multilayer pharmaceutical form according to claim 1, wherein the substance having a modulating effect has a molecular weight below 500 and is in solid form and is ionogenic.

9. The multilayer pharmaceutical form according to claim 7, wherein the substance having a modulating effect is soluble in water.

10. The multilayer pharmaceutical form according to claim 7, wherein the substance having a modulating effect is an organic acid or the salt of an organic or inorganic acid.

11. The multilayer pharmaceutical form according to claim 1, wherein the substance having a modulating effect is succinic acid, citric acid, tartaric acid, laurylsulphuric acid, a salt of these acids or a salt of the following anions: taurochlolate and other cholates, chlorides, acetates, lactates, phosphates and/or sulphates.

12. The multilayer pharmaceutical form according to claim 1, wherein the active ingredient layer c) comprises metoprolol succinate, and the active ingredient release measured according to USP, 100 rpm, pH 6.8, is slower in the 2 hour intervals up to the fourth hour than in the 2 hour intervals from the fourth to the tenth hour.

13. The multilayer pharmaceutical form according to claim 1, wherein the active ingredient layer c) comprises terbutaline sulphate, and the active ingredient release measured according to USP, 100 rpm, pH 6.8 is approximately constant in 2 hour intervals up to the twelfth hour.

14. A process for producing a multilayer pharmaceutical form according to claim 1 in a manner known per se by means of pharmaceutically customary processes such as direct compression, compression of dry, wet or sintered granules, extrusion and subsequent rounding off, wet or dry granulation or direct pelleting or by binding of powders (powder layering) onto active ingredient-free beads or neutral cores (nonpareilles) or active ingredient-containing particles or by means of spraying processes or fluidized bed granulation.

15. Use of a multilayer pharmaceutical form according to claim 1 as ingredient of a multiparticulate pharmaceutical form, of pellet-containing tablets, minitablets, capsules, sachets, effervescent tablets or powders for reconstitution.
